(19) United States
(12) Patent Application Publication
(10) Pub. No.: US 2026/0047836 A1
(43) Pub. Date: Feb. 19, 2026

(54) CLIPS WITH COUPLERS FOR LEFT ATRIAL APPENDAGE CLOSURE

(71) Applicant: EDWARDS LIFESCIENCES CORPORATION, Irvine, CA (US)

(72) Inventors: Cindy Woo, Irvine, CA (US); Harvey H. Chen, Irvine, CA (US)

(21) Appl. No.: 19/369,981

(22) Filed: Oct. 27, 2025

Related U.S. Application Data
(63) Continuation of application No. PCT/US2024/027417, filed on May 2, 2024.
(60) Provisional application No. 63/499,964, filed on May 3, 2023.

(51) Int. Cl. A61B 17/00

(52) U.S. Cl. CPC A61B 17/0057; A61B 2017/00477; A61B 2017/00584; A61B 2017/00623; A61B 2017/00632

(57) ABSTRACT

A clip may be configured to close the portion of the heart, to reduce blood flow therethrough as well as passage of clots or other undesired materials. The clip may be configured to close the left atrial appendage (LAA). The closure of the LAA may reduce the possibility of stroke or other maladies stemming from fluid flow with the LAA.

### CROSS REFERENCE TO RELATED APPLICATIONS

This application is a continuation of International Application No. PCT/US2024/027417, filed May 2, 2024, which claims the benefit of U.S. Patent Application No. 63/499,964, filed May 3, 2023, the entire disclosures all of which are incorporated by reference for all purposes.

### BACKGROUND

### Field

Various examples disclosed herein relate generally to clips for medical implementation. Some examples relate to clips for a left atrial appendage (LAA).

### Background

Cardiac arrhythmias are abnormal heart rhythms that can cause the heart to pump blood less effectively. Atrial fibrillation (AF) is one of the most common heart arrhythmia conditions. AF causes the left atrium to beat irregularly and reduces the efficiency of the “atrial kick” that helps to move blood into the left ventricle.

The left atrial appendage (LAA) is a muscular pouch located high on the free wall of the left atrium. The anatomy of the LAA is such that blood has a tendency to stagnate and form clots within the LAA. As blood flow is reduced with the progression of AF, the potential for clot formation increases tremendously.

Clots formed in the LAA can embolize into the bloodstream and move into the brain, where they can become lodged and eventually lead to stroke. It may be beneficial to close or occlude the LAA, to reduce the possibility of clots or other undesired materials from passing into the left atrium and into the bloodstream.

Left atrial appendage closure (also known as LAA closure or LAAC) is a minimally invasive procedure that is used to reduce the risk of stroke that comes as a result of atrial fibrillation.

### SUMMARY

Systems, apparatuses, and methods disclosed herein may be directed to clips for medical implementation, including clips for a portion of a heart. The clips may be configured to close the portion of the heart, to reduce blood flow therethrough as well as passage of clots or other undesired materials. In examples, the clips may be configured to close the left atrial appendage (LAA). The closure of the LAA may reduce the possibility of stroke or other maladies stemming from fluid flow with the LAA. In examples, the clips may be positioned exterior of the LAA, to extend over an outer surface of the LAA for closure.

In aspects, a clip for a portion of a heart. The clip may include a first jaw and a second jaw forming a distal end portion of the clip and a proximal end portion of the clip, the first jaw and the second jaw configured to compress the portion of the heart. The clip may include a coupler positioned at the proximal end portion of the clip, the coupler configured to couple with an engagement portion of an expansion apparatus for the clip.

In aspects, a method comprising deploying a clip to close a portion of a heart. The clip may include a first jaw and a second jaw forming a distal end portion of the clip and a proximal end portion of the clip, the first jaw and the second jaw configured to compress the portion of the heart. The clip may include a coupler positioned at the proximal end portion of the clip, the coupler configured to couple with an engagement portion of an expansion apparatus for the clip.

In aspects, an expansion apparatus for a left atrial appendage clip extending along an axis. The expansion apparatus may include an elongate shaft having a first end portion and a second end portion. The expansion apparatus may include an engagement portion positioned at the first end portion of the elongate shaft and having a coupler configured to axially engage with a coupler of the left atrial appendage clip. The expansion apparatus may include a handle positioned at the second end portion of the elongate shaft.

In aspects, a method comprising utilizing an expansion apparatus to deploy or capture a left atrial appendage clip. The expansion apparatus may include an elongate shaft having a first end portion and a second end portion. The expansion apparatus may include an engagement portion positioned at the first end portion of the elongate shaft and having a coupler configured to axially engage with a coupler of the left atrial appendage clip. The expansion apparatus may include a handle positioned at the second end portion of the elongate shaft.

### BRIEF DESCRIPTION OF THE DRAWINGS

FIG. 1 shows a cross sectional schematic view of a portion of a heart.

FIG. 2 illustrates a perspective view of a clip.

FIG. 3 illustrates a perspective view of the clip shown in FIG. 2 from an opposite side of the clip than shown in FIG. 2.

FIG. 4 illustrates a cross sectional view of the clip shown in FIG. 2.

FIG. 5 illustrates a cross sectional view of the clip shown in FIG. 2 at a view orthogonal to the view shown in FIG. 4.

FIG. 6 illustrates a perspective view of an expansion apparatus.

FIG. 7 illustrates a detail perspective view of an engagement portion of the expansion apparatus shown in FIG. 6.

FIG. 8 illustrates a cross sectional view of the shaft of the expansion apparatus shown in FIG. 6.

FIG. 9 illustrates a side schematic view of an expansion apparatus approaching a deployed clip.

FIG. 10 illustrates a side schematic view of an expansion apparatus engaging a deployed clip.

FIG. 11 illustrates a side schematic view of an expansion apparatus engaging a deployed clip.

FIG. 12 illustrates a side schematic view of an expansion apparatus expanding a deployed clip.

FIG. 13 illustrates a side schematic view of an expansion apparatus approaching a deployed clip.

FIG. 14 illustrates a side schematic view of an expansion apparatus approaching a deployed clip with a portion of the expansion apparatus shown in cross section.

### DETAILED DESCRIPTION

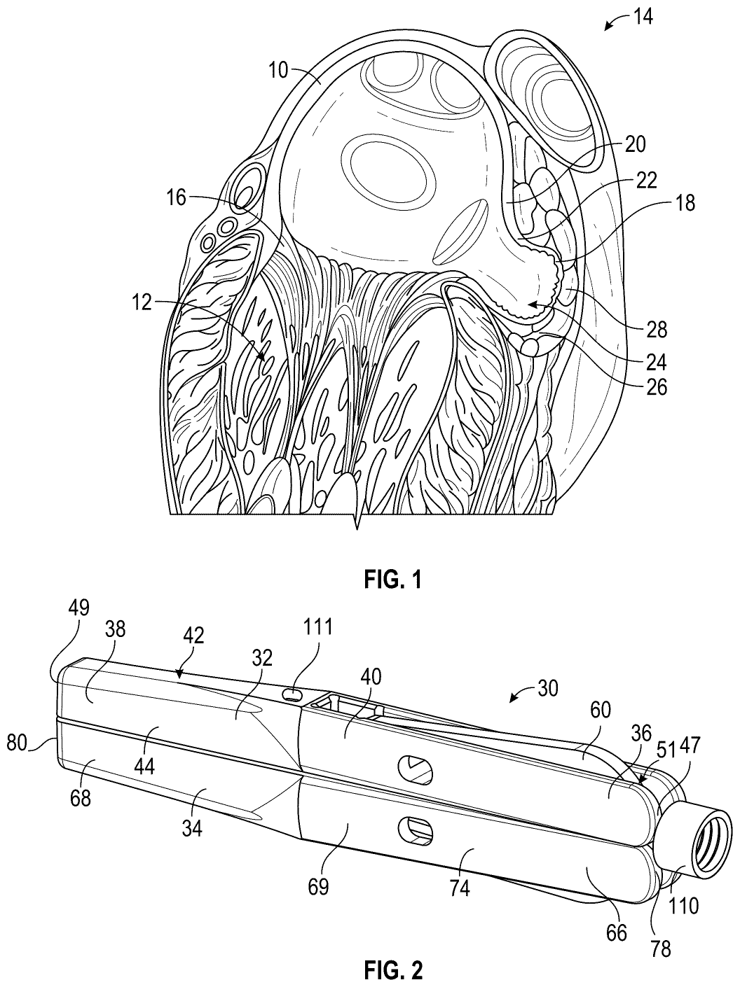

FIG. 1 illustrates a cross sectional view of a left atrium 10 and a left ventricle 12 of an individual's heart 14. The left atrium 10 is configured to fill with blood to pass into the left ventricle 12 via the mitral valve 16 during the cardiac cycle. The left atrial appendage (LAA) 18 protrudes from the outer wall 20 of the left atrium 10 and includes an ostium 22 and a cavity 24 extending from the ostium 22. A LAA wall 26 may surround the cavity 24 and may form an outer surface 28 of the LAA 18. The LAA 18 has a pouch shape extending from the left atrium 10. The cavity 24 may be configured to fill with blood, allowing the LAA to serve as a decompression chamber during systole and when pressure otherwise increases in the left atrium 10.

In certain individuals, blood may stagnate and form clots within the LAA 18. Clots or other undesired materials stemming from the LAA 18 may travel into the bloodstream, producing a variety of maladies including strokes. It may thus be beneficial to close the LAA 18, to reduce the possibility of clots or other undesired material from producing such maladies.

FIG. 2 illustrates an example of a clip 30 that may be utilized for a portion of a heart. The clip 30 may be configured to close a LAA 18, to reduce the possibility of clots or other undesired materials stemming from the LAA 18 from traveling into the bloodstream.

The clip 30 may include a first jaw 32 and a second jaw 34. The first jaw 32 may extend from a first end portion 36 to a second end portion 38 along a length of the first jaw 32. The first jaw 32 may have an elongate shape. The first jaw 32 may be configured as an elongate beam. The first jaw 32 may have a central portion 40 between the first end portion 36 and the second end portion 38. The first jaw 32 may extend along a longitudinal axis of the clip 30.

The first end portion 36 may comprise a proximal end portion of the clip 30. The second end portion 38 may comprise a distal end portion of the clip 30.

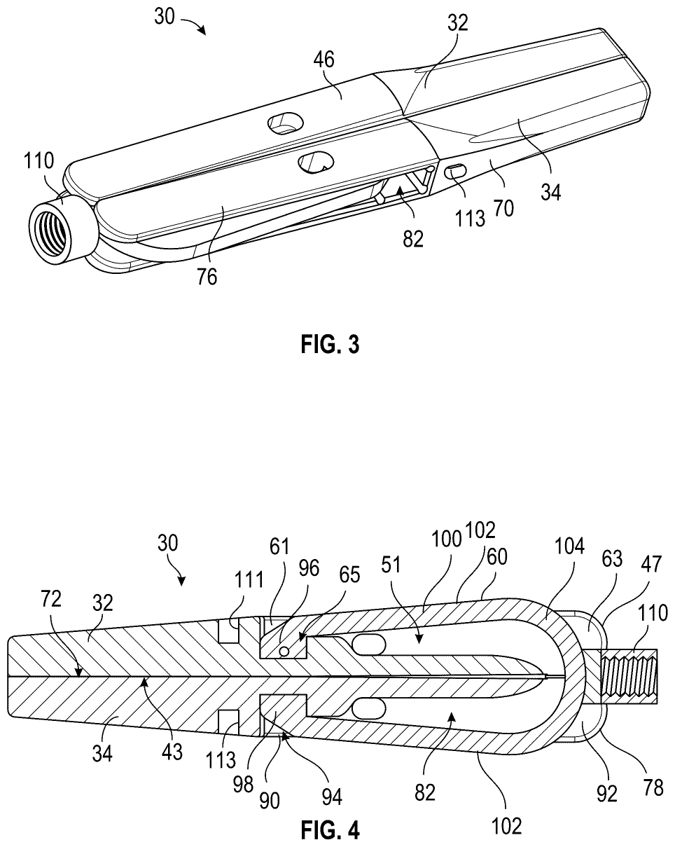

The first jaw 32 may include an outer surface 42, a compression surface 43 (marked in FIG. 4), and two side surfaces 44, 46 (with side surface 46 shown in FIG. 3) each extending from the compression surface 43 to the outer surface 42. The compression surface 43 may face opposite the outer surface 42 and towards the second jaw 34.

The first jaw 32 may include a first end surface 47 positioned at the first end portion 36 of the first jaw 32. The first jaw 32 may include a second end surface 49 positioned at the second end portion 38 of the first jaw 32. The first end surface 47 may comprise a proximal end surface of the first jaw 32 and the second end surface 49 may comprise a distal end surface of the first jaw 32.

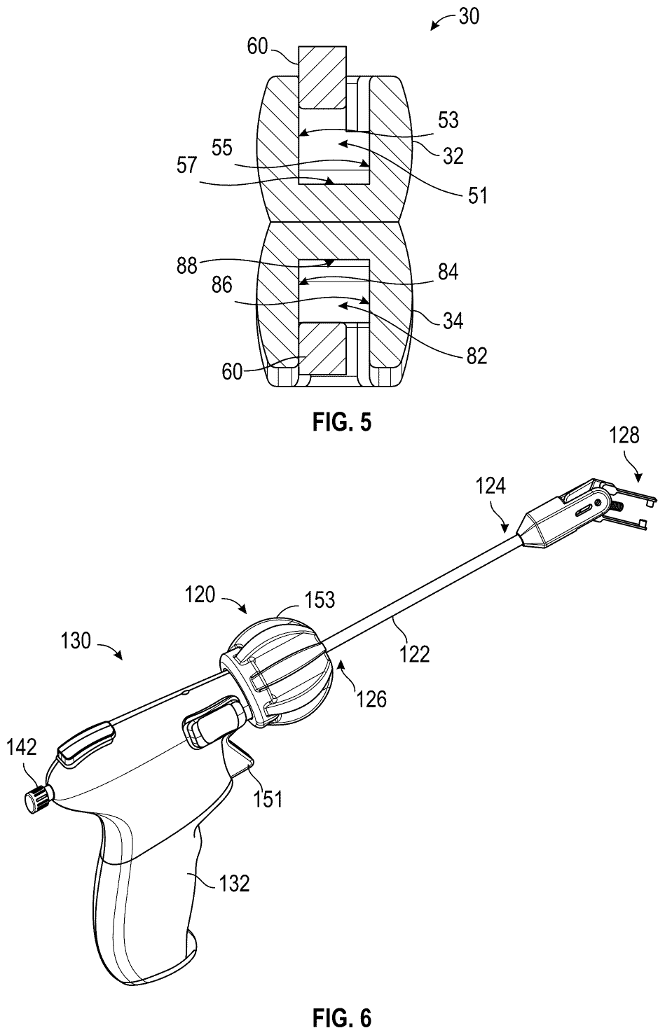

The outer surface 42 of the first jaw 32 may include a channel 51 (marked in FIG. 5). FIG. 5 illustrates a cross sectional view of the clip 30. Referring to FIG. 5, the channel 51 may include a first side wall 53, a second side wall 55, and a lower side wall 57 of the channel 51. The upper portion of the channel 51 may remain open for a spring 60 to pass through. The channel 51 may extend from the central portion 40 of the first jaw 32 to the first end portion 36 of the first jaw 32. Referring to the cross sectional view of FIG. 4, the channel 51 may include a first end 61 and a second end 63, with the second end 63 being opened to allow for the spring 60 to pass through.

A coupler 65 may be positioned at the first end 61 of the channel 51 and configured to receive an end of the spring 60. The coupler 65 may comprise a cavity in the first jaw 32 or may have another configuration as desired.

The second jaw 34 may be configured similarly as the first jaw 32 and may include similar features.

The second jaw 34 may extend from a first end portion 66 to a second end portion 68 along a length of the second jaw 34. The second jaw 34 may have an elongate shape. The second jaw 34 may be configured as an elongate beam. The second jaw 34 may have a central portion 69 between the first end portion 66 and the second end portion 68. The second jaw 34 may extend along a longitudinal axis of the clip 30.

The first end portion 66 may comprise a proximal end portion of the clip 30. The second end portion 68 may comprise a distal end portion of the clip 30.

The second jaw 34 may include an outer surface 70 (marked in FIG. 3), a compression surface 72 (marked in FIG. 4), and two side surfaces 74, 76 (with side surface 76 shown in FIG. 3) each extending from the compression surface 72 to the outer surface 70. The compression surface 72 may face opposite the outer surface 70 and towards the first jaw 32.

The second jaw 34 may include a first end surface 78 positioned at the first end portion 66 of the second jaw 34. The second jaw 34 may include a second end surface 80 positioned at the second end portion 68 of the second jaw 34. The first end surface 78 may comprise a proximal end surface of the second jaw 34 and the second end surface 80 may comprise a distal end surface of the second jaw 34.

The outer surface 70 of the second jaw 34 may include a channel 82 (marked in FIG. 5). FIG. 5 illustrates a cross sectional view of the clip 30. Referring to FIG. 5, the channel 82 may include a first side wall 84, a second side wall 86, and a lower side wall 88 of the channel 82. The upper portion of the channel 82 may remain open for the spring 60 to pass through. The channel 82 may extend from the central portion 69 of the second jaw 34 to the first end portion 66 of the second jaw 34. Referring to the cross sectional view of FIG. 4, the channel 82 may include a first end 90 and a second end 92, with the second end 92 being opened to allow for the spring 60 to pass through.

A coupler 94 may be positioned at the first end 90 of the channel 82 and configured to receive an end of the spring 60. The coupler 94 may comprise a cavity in the second jaw 34 or may have another configuration as desired.

The first jaw 32 and the second jaw 34 may form a distal end portion 38, 68 of the clip 30 and a proximal end portion 36, 66 of the clip 30.

The spring 60 may be configured to compress the first jaw 32 towards the second jaw 34 to compress a portion of the LAA between the first jaw 32 and the second jaw 34. The spring 60 may extend over the outer surface 42 of the first jaw 32 and the outer surface 70 of the second jaw 34 and may be configured to force the first jaw 32 and the second jaw 34 together to compress the portion of the heart between the compression surface 43 of the first jaw 32 (marked in FIG. 4) and the compression surface 72 of the second jaw 34 (marked in FIG. 4). Referring to FIG. 4, the spring 60 may have a “C” shape with a first end 96 and a second end 98 and a loop 100 coupled to the first end 96 and the second end 98. The loop 100 may include straight portions 102 coupled to a curved portion 104 forming the curve of the “C” shape. The first end 96 of the spring 60 may be coupled to the central portion 40 of the first jaw 32 (marked in FIG. 2) and the second end 98 of the spring 60 may be coupled to the central portion 69 of the second jaw 34 (marked in FIG. 2), and the loop 100 may extend towards the first end portion 36 of the first jaw 32 and the first end portion 66 of the second jaw 34.

The loop 100 may extend within the channel 51 of the first jaw 32 and the channel 82 of the second jaw 34. The loop 100 may be positioned within the channel 51 of the first jaw 32 and the channel 82 of the second jaw 34 between the respective side walls 53, 55 of the channel 51 and the side walls 84, 86 of the channel 82 (marked in FIG. 5). The loop 100 may be spaced from the respective lower walls 57, 88 of the channels 51, 82 such that the loop 100 may move towards the respective lower walls 57, 88 upon the clip 30 being in an opened state. The spring 60, for example, may pivot with respect to the lower walls 57, 88 upon the clip 30 being in an opened state.

The position of the loop 100 within the channels 51, 82 may allow for expansion of the clip 30. The position of the loop 100 within the channels 51, 82 may reduce the possibility of twisting of the spring 60 during opening or closing of the clip 30.

The loop 100 may reside within the channels 51, 82 continuously and may reduce the possibility of the jaws 32, 34 from moving distally and side to side.

The spring 60 may extend within the plane of movement of the first jaw 32 and the second jaw 34.

The spring 60 may be configured to allow the clip 30 to move from an opened state to a closed state, yet force the clip 30 towards the closed state. The spring 60 accordingly may provide a force that moves the compression surfaces 43, 72 of the first jaw 32 and second jaw 34 towards each other to compress a portion of the heart therein. The spring 60 may be configured to keep the jaws 32, 34 under positive compression at all times, even at rest. The spring 60 may have a “C” shape to allow the second end portions 38, 68 of the first jaw 32 and the second jaw 34 respectively to form an axial opening 106 (marked in FIG. 12) for a space 108 (marked in FIG. 12) between the first jaw 32 and the second jaw 34 for receiving the portion of the heart. The space 108 may comprise a compression channel between the first jaw 32 and the second jaw 34. The axial opening 106 may comprise an opening at the second end portions 38, 68 of the first jaw 32 and second jaw 34. The clip 30 may be positioned in an opened state and with the portion of the heart slid through the axial opening 106 and into the space 108. In examples, other methods of entry into the space 108 may be provided (e.g., along an axis of the LAA).

The second end portion 38 of the first jaw 32 and the second end portion 68 of the second jaw 34 may form an open end of the clip 30. The compression channel between the first jaw 32 and the second jaw 34 may have an opening at the second end portions 38, 68 of the clip 30. The first end portion 36 of the first jaw 32 and the first end portion 66 of the second jaw 34 may form a closed end of the clip 30.

The loop 100 of the spring 60 may form a boundary of the space 108 between the first jaw 32 and the second jaw 34. The compression channel may be closed at the first end portions 36, 66 of the first jaw 32 and second jaw 34. The loop 100 of the spring 60 may close the space 108 at the first end portion 36 of the first jaw 32 and the first end portion 66 of the second jaw 34. The loop 100 accordingly may prevent the clip 30 from sliding distally with respect to the LAA 18 upon deployment, and may prevent the tissue of the LAA 18 from protruding further than the loop 100 upon compression of the LAA 18.

FIG. 3 illustrates a perspective view of the clip 30 from an opposite side of the clip 30 than shown in FIG. 2. FIG. 4 illustrates a cross sectional view of the clip 30.

In examples, a coupler 110 may be positioned at the proximal end portion of the clip 30. The coupler 110 may be positioned at the proximal end portions of the first jaw 32 and the second jaw 34. The coupler 110 may be configured to couple with an engagement portion of an expansion apparatus for the clip 30. The coupler 110 may be configured to axially engage a coupler of the expansion apparatus. The coupler 110 may face towards a proximal direction.

The coupler 110 may be utilized for capture of the clip 30 in a configuration in which the clip 30 has been deployed to a portion of the heart and should be captured for repositioning or removal. In examples, the coupler 110 may be utilized in a deployment procedure or a capture procedure of the clip 30.

Referring to FIG. 4, the coupler 110 may be coupled to the spring 60 at the loop 100 or curved portion 104 of the spring 60. The jaws 32, 34 may be configured to open and close independent of the position of the coupler 110. In examples, the coupler 110 may be positioned on one or more of the jaws 32, 34. The coupler 110 may be positioned at the respective first end surfaces 47, 78 of the first jaw 32 and the second jaw 34.

In examples, the coupler 110 may be configured to rotatably engage with the engagement portion of an engagement apparatus. The coupler 110, for example, may threadably engage with the engagement portion of the engagement apparatus. The coupler 110 may comprise a threaded body that may include a cavity (e.g., a female threaded body) for receiving a threaded coupler of the expansion apparatus. In examples, other configurations of the coupler 110 may be utilized, for example, the coupler 110 may comprise a protrusion have threading on an outside surface of the coupler 110 (e.g., a male threaded body). The corresponding coupler of the engagement portion may have a cavity for receiving the protrusion. Other forms of coupling may be utilized in examples.

The clip 30 may utilize other couplers in examples. For example, the clip 30 may include a coupler 111 on the first jaw 32 as shown in FIG. 2 for example. The coupler 111 may be positioned on the outer surface 42 of the first jaw 32 or in another position as desired. The coupler 111 may comprise a recess or may have another configuration in examples. The clip may include a coupler 113 on the second jaw 34 as shown in FIG. 3 for example. The coupler 113 may be positioned on the outer surface 70 of the second jaw 34 or in another position as desired. The coupler 113 may comprise a recess or may have another configuration in examples. The couplers 111, 113 may each be configured to couple with an engagement portion of an expansion apparatus in examples.

The coupler 110 may be positioned proximal of each of the couplers 111, 113 in examples.

FIG. 6 illustrates a perspective view of an expansion apparatus 120 that may be utilized in examples. The expansion apparatus 120 may include an elongate shaft 122. The elongate shaft 122 may have a first end portion 124 or distal end portion and a second end portion 126 or proximal end portion. The elongate shaft 122 may comprise a rigid shaft or may comprise a flexible shaft in examples. The elongate shaft 122 may be configured for one or more components of an actuator assembly to pass therethrough, to extend to the engagement portion 128 in examples.

The expansion apparatus 120 may include a handle 130 in examples. The handle 130 may be positioned at the second end portion 126 or proximal end portion of the elongate shaft 122. The handle 130 may include a grip portion 132 for a user to grip in examples.

The expansion apparatus 120 may include an engagement portion 128. The engagement portion 128 may be configured to engage the clip 30 in examples. The engagement portion 128 may be positioned at the first end portion 124 of the elongate shaft 122 in examples.

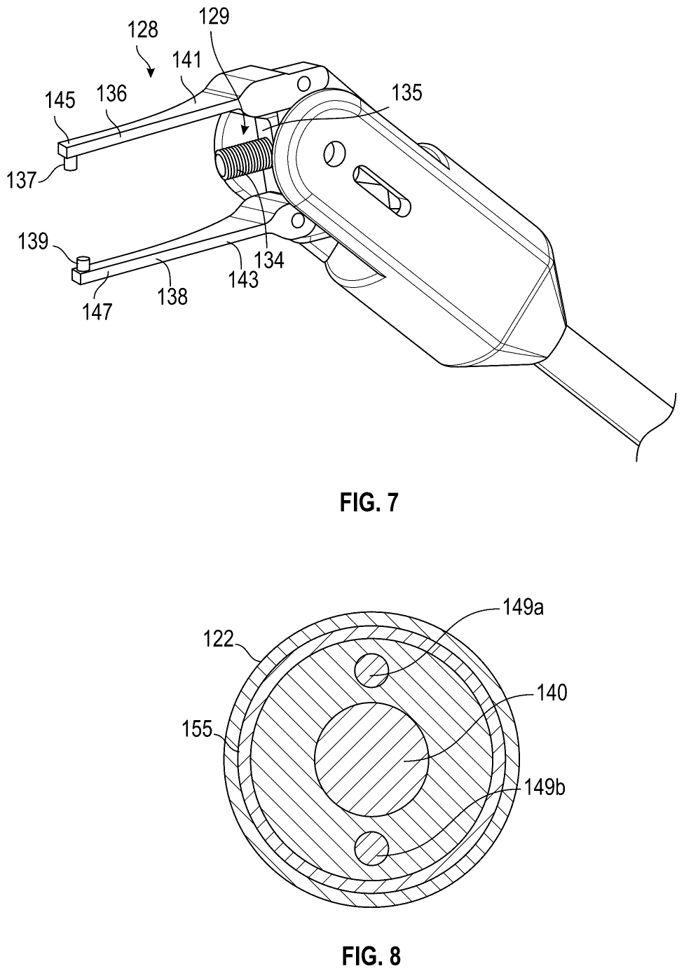

FIG. 7 illustrates a perspective view of the engagement portion 128. The engagement portion 128 may include a coupler 134 that may be configured to couple with the coupler 110 shown in FIG. 2. The coupler 134 may comprise an axial coupler that may axially engage with the coupler 110 of the clip 30.

The coupler 134 may comprise a protrusion. The coupler 134 may comprise a threaded coupler (e.g., a male threaded coupler) configured to threadably engage with the coupler 110 (e.g., a female threaded coupler) of the clip 30. The coupler 134 may be configured to rotatably engage with the coupler 110 shown in FIG. 2. The coupler 134 may have other configurations (e.g., a female threaded coupler with the coupler 110 comprising a male threaded coupler) in examples as desired.

The engagement portion 128 may include a central portion 129. The coupler 134 may be positioned upon the central portion 129. The coupler 134 may be configured to rotate relative to a base 135 of the engagement portion 128 to axially engage the coupler 110 of the clip 30. Other positions of the coupler 134 may be utilized in examples.

The engagement portion 128 may include arms 136, 138 that may be utilized to engage with the clip 30. The arms 136, 138, for example may engage with the couplers 111, 113 positioned on the respective jaws 32, 34 as marked in FIG. 4. The arm 136 may include a coupler 137 configured to engage the coupler 111 on the first jaw 32, and the arm 138 may include a coupler 139 configured to engage the coupler 113 on the second jaw 34.

The arms 136, 138 may each have a respective proximal end portion 141, 143 and a distal end portion 145, 147. The proximal end portions 141, 143 may be coupled to the central portion 129. The couplers 137, 139 may be positioned on the respective distal end portions 145, 147 of the arms 136, 138.

The arms 136, 138 may be pivotally coupled to the central portion 129 and configured to move relative to each other to open or close the clip 30. The arms 136, 138 may be configured to pivot relative to the central portion 129. The arms 136, 138 may move away from each other to open the clip 30, or may move towards each other to close the clip 30.

An actuator assembly may be utilized to actuate the coupler 134 or one or more of the arms 136, 138. The actuator assembly may have a variety of forms in examples.

The actuator assembly may include a drive shaft 140 in examples. A cross sectional view of the elongate shaft 122 is shown in FIG. 8. The drive shaft 140 may extend along the length of the elongate shaft 122. The drive shaft 140 may be utilized to rotate the coupler 134 relative to the base 135. The drive shaft 140 may be actuated by a control device 142 as shown in FIG. 6 for example. The control device 142 may be configured for a user to operate to actuate the coupler 134. The control device 142 may comprise a knob configured to be rotated to rotate the coupler 134 or may have another configuration in examples. The rotation of the control device 142 may rotate the drive shaft 140 and accordingly the coupler 134.

The actuator assembly may be utilized to control the arms 136, 138 in examples. For example, the actuator assembly may include control bodies 149a, b (marked in the cross sectional view of FIG. 8) that may be actuated by a control device 151. The control bodies 149a, b may move proximally or distally to control the opening and closing of the respective arms 136, 138. The control device 151 may comprise a trigger or push button or may have another form in examples.

In examples, the engagement portion 128 may be configured to rotate relative to the elongate shaft 122 or with the elongate shaft 122. A control shaft 155, as marked in FIG. 8, may extend to the engagement portion 128 and may be configured to be rotated to correspondingly rotate the engagement portion 128. The control shaft 155 may be controlled by a control device 153 (as marked in FIG. 6). The control device 153 may comprise a rotatable knob or may have another configuration in examples.

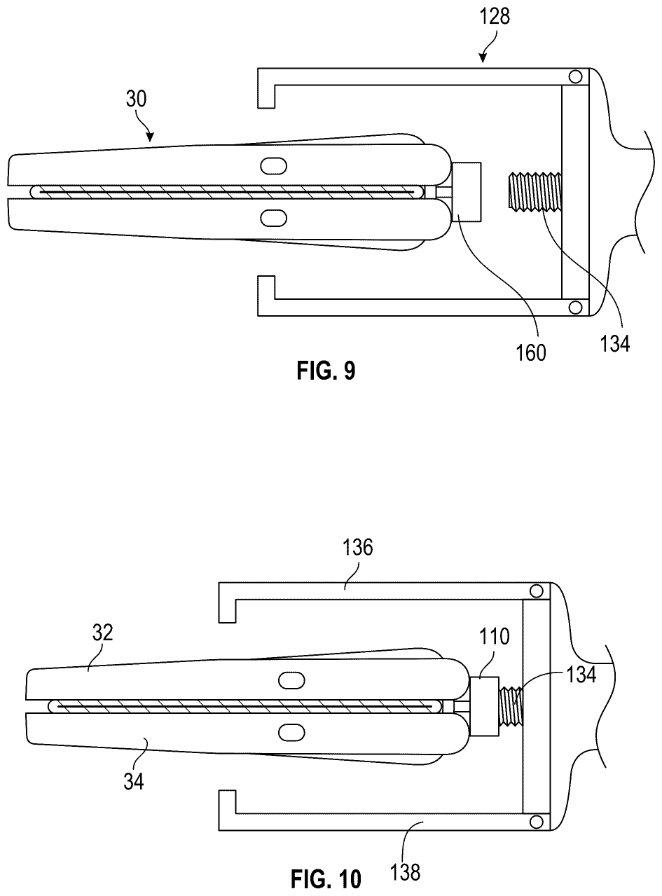

FIGS. 9-12 illustrate an exemplary operation of the expansion apparatus 120. The clip 30 may be deployed to the LAA as shown in FIG. 9. The engagement portion 128 of the expansion apparatus 120 may approach the deployed clip 30.

Referring to FIG. 10, the coupler 134 of the expansion apparatus 120 may engage the coupler 110 of the clip 30. The coupler 134 may screw into the coupler 110 or other forms of engagement may be utilized. The coupler 134 may be secured in position relative to the clip 30. The connection between the coupler 134 of the expansion apparatus 120 and the coupler 110 of the clip 30 may allow for improved positioning of the engagement portion 128 of the expansion apparatus 120 relative to the clip 30.

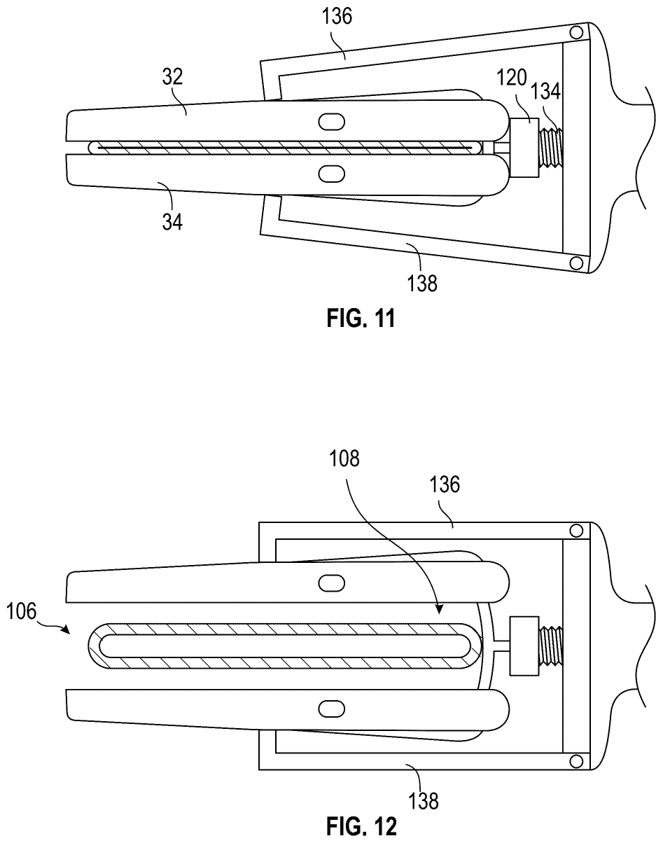

The arms 136, 138 of the expansion apparatus 120 may be pivoted to connect with the jaws 32, 34. The arms 136, 138 may insert into the couplers 111, 113 as marked in FIG. 4 for example. FIG. 11 illustrates such an arrangement.

The arms 136, 138 may be utilized to open the clip 30. FIG. 12 illustrates the arms 136, 138 having been expanded to open the clip 30. The captured clip 30 may be removed or repositioned as desired. The couplers 111, 113 may be relatively moved away from each other to move the clip 30 to an opened state. The couplers 111, 113 may be configured to be relatively moved towards each other to move the clip 30 to a closed state.

In examples, the expansion apparatus 120 may be utilized for an initial deployment of the clip 30. A reverse operation than shown in FIGS. 9-12 may be utilized, among other methods.

Variations of the expansion apparatus 120 or clip 30 may be utilized.

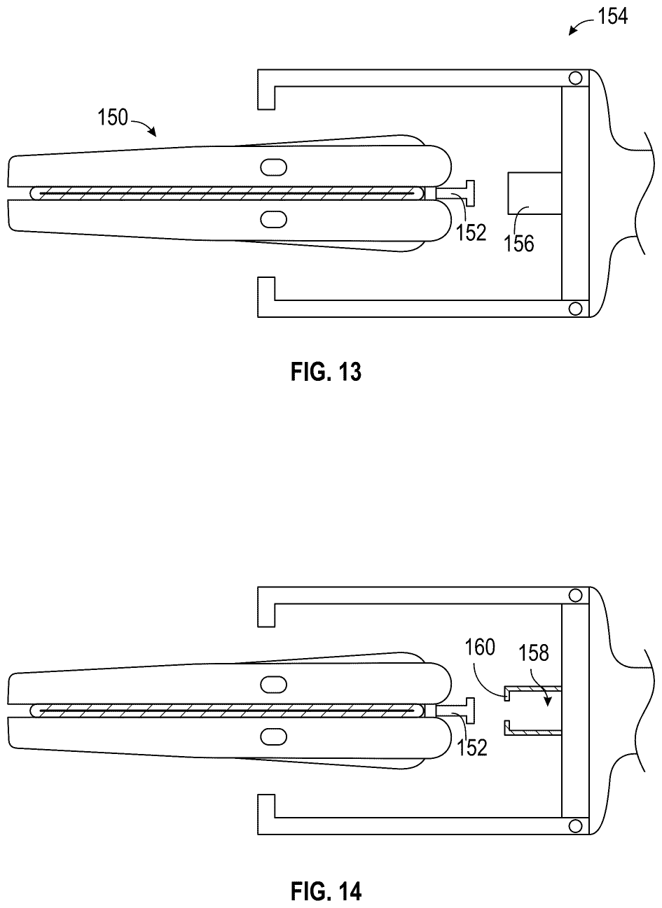

FIGS. 13 and 14, for example, illustrate a variation in which a clip 150 may include an axially extending coupler in the form of a quick lock device 152. The expansion apparatus 154 may include a coupler 156 that may be configured to engage the quick lock device 152. The coupler 156, for example, may comprise a cavity 158 with a lip 160 for engaging the quick lock device 152 as shown in FIG. 14 for example. The features of the clip 30 may otherwise be utilized with the clip 150, and the features of the expansion apparatus 120 may otherwise be utilized with the expansion apparatus 154.

Other configurations of clips and expansion apparatuses may be utilized in examples as desired.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The clips may be utilized solely or in methods disclosed herein, or in combination with other devices disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosure. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A clip for a portion of a heart, the clip comprising:
a first jaw and a second jaw forming a distal end portion of the clip and a proximal end portion of the clip, the first jaw and the second jaw configured to compress the portion of the heart;
a first coupler positioned at the proximal end portion of the clip, the first coupler configured to couple with an engagement portion of an expansion apparatus for the clip;
a second coupler positioned on the first jaw and configured to couple with the engagement portion of the expansion apparatus; and
a third coupler positioned on the second jaw and configured to couple with the engagement portion of the expansion apparatus, and
wherein the second coupler is configured to be relatively moved away from the third coupler to move the clip to an opened state.

2. The clip of claim 1, wherein the first jaw and the second jaw extend along a longitudinal axis of the clip, and the first coupler is configured to be engaged axially by the engagement portion of the expansion apparatus.

3. The clip of claim 1, wherein the first coupler is configured to rotatably engage with the engagement portion of the expansion apparatus.

4. The clip of claim 1, wherein the first coupler is configured to threadably engage with the engagement portion of the expansion apparatus.

5. The clip of claim 1, wherein the first coupler is configured to face towards a proximal direction.

6. The clip of claim 1, wherein the second coupler is configured to be relatively moved towards the third coupler to move the clip to a closed state.

7. The clip of claim 1, wherein the first coupler is positioned proximal of the second coupler and the third coupler.

8. The clip of claim 1, wherein the first jaw includes:
a first end surface positioned at the proximal end portion of the clip;
a second end surface positioned at the distal end portion of the clip;
a compression surface configured to face towards the second jaw;
an outer surface facing opposite the compression surface of the first jaw;
a first side surface extending from the compression surface to the outer surface of the first jaw; and
a second side surface extending from the compression surface to the outer surface of the first jaw and facing opposite the first side surface, and
the first coupler is positioned at the first end surface of the first jaw.

9. The clip of claim 8, wherein the second coupler is positioned on the outer surface of the first jaw.

10. The clip of claim 8, wherein the second jaw includes:
a first end surface positioned at the proximal end portion of the clip;
a second end surface positioned at the distal end portion of the clip;
a compression surface configured to face towards the first jaw;
an outer surface facing opposite the compression surface of the second jaw;
a first side surface extending from the compression surface of the second jaw to the outer surface of the second jaw; and
a second side surface extending from the compression surface of the second jaw to the outer surface of the second jaw and facing opposite the first side surface, and
the first coupler is positioned at the first end surface of the second jaw.

11. The clip of claim 10, wherein the third coupler is positioned on the outer surface of the second jaw.

12. The clip of claim 1, further comprising a spring configured to force the first jaw and the second jaw together to compress the portion of the heart with the first jaw and the second jaw.

13. The clip of claim 12, wherein the spring includes a loop extending towards the proximal end portion of the clip.

14. The clip of claim 12, wherein the first jaw includes a channel, the spring being positioned within the channel.

15. The clip of claim 14, wherein the channel includes a first side wall, a second side wall, a lower side wall, and an upper portion that is open for the spring to pass through.

16. The clip of claim 14, wherein the channel includes a first end and a second end, and the second end is opened to allow the spring to pass through.

17. The clip of claim 16, wherein a coupler is positioned at the first end of the channel and configured to receive an end of the spring.

18. The clip of claim 1, wherein the first jaw and the second jaw form a compression channel between the first jaw and the second jaw, the compression channel having an opening at the distal end portion of the clip, and the compression channel being closed at the proximal end portion of the clip.

19. The clip of claim 18, further comprising a spring closing the compression channel at the proximal end portion of the clip.

20. The clip of claim 1, wherein the clip is configured to occlude a left atrial appendage.
